(19) United States
(12) Patent Application Publication
(10) Pub. No.: US 2003/0170239 A1
(43) Pub. Date: Sep. 11, 2003

(54) Immunotherapeutic method to prevent islet cell rejection

(76) Inventors: Bernhard J. Hering, Minnetonka, MN (US); Martin Wijkstrom, St. Paul, MN (US); Nicole Kirchhof, Minnetonka, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/306,860

(22) Filed: Nov. 26, 2002

Related U.S. Application Data
(63) Continuation of application No. PCT/US01/18001, filed on Jun. 1, 2001.
(60) Provisional application No. 60/208,725, filed on Jun. 2, 2000.

(51) Int. Cl. A61K 39/395

(52) U.S. Cl. 424/144.1

(57) ABSTRACT

A method for the prevention or reversal of islet cell transplant rejection, or for therapy for autoimmune diseases, is provided comprising administering compounds such as monoclonal antibodies, that bind specifically to CD40L and the CD4 receptor.

### BACKGROUND OF THE INVENTION

Diabetes affects approximately 16 million people in the United States, including over one million patients with type 1 (insulin dependent) diabetes, and continues to be a therapeutic challenge. More than 14% of U.S. health care dollars are spent on diabetes, a total of $122 billion in 1994 alone. However, diabetes remains one of the leading causes of death by disease, and is the leading cause of blindness, kidney failure and non-traumatic amputations.

The principal determinant of the risk of the devastating complications of diabetes is the total lifetime exposure to elevated blood glucose levels. Therefore, establishing safe and effective methods of achieving and maintaining normoglycemia will have substantial implications for the health and quality of life of individuals with diabetes. The Diabetes Control and Complications Trial (DCCT) demonstrated that in a setting of a qualified diabetes control care team, intensive control with near normalization of glycemia could be achieved and sustained for several years. However, such treatment is labor intensive, difficult to implement for many patients, and limited by the accompanying increased frequency of severe hypoglycemia. Today, the only way to restore normal blood glucose levels without the associated risk of hypoglycemia is to replace the patient's islets of Langerhans. This may be achieved, for example, by the transplantation of a whole pancreas, or, by the injection of islets of Langerhans.

Successful whole pancreas transplantation induces euglycemia in nearly all patients, but surgical risk, complications associated with the exocrine portion of the pancreas, and organ availability limit such transplants to a minority of patients. Islet cell transplantation could significantly reduce risk and morbidity, but organ availability also restricts the practice of islet transplantation.

Xenogeneic islet cell transplantation has been problematic as well. In nude mice and rats, islet xenografts are characterized by the progressive infiltration of inflammatory cells. Fetal and adult islet xenografts in mice and rats with ongoing rejection exhibit a cellular distribution in which macrophages are centrally arranged around the collapsing endocrine cells and T cells surround the entire graft area, a pattern reminiscent of delayed type hypersensitivity reactions. In non-human primate recipients, the rejection process of islet xenografts is more vigorous and is dominated by a massive infiltration of T cells. Immunohistochemical studies of immunosuppressed primates have shown that macrophages are the main cellular subtype infiltrating islet xenografts. Data suggest islet xenografts succumb to cell-mediated rejection in a T-cell dependent manner.

The T-cell mediated immune response is initially triggered by helper T-cells (Tₕ) which are capable of recognizing specific antigens. When one of these Tₕ cells recognizes an antigen present on the surface of an antigen presenting cell (APC) or a macrophage in the form of an antigen-MHC complex, the Tₕ cell is stimulated to produce IL-2 by signals emanating from the antigen-specific T-cell receptor, co-receptors, and IL-1 secreted by the APC or macrophage. The Tₕ cells then proliferate, resulting in a large population of T-cells which are clonally selected to recognize a particular antigen. T-cell activation may also stimulate B-cell activation and nonspecific macrophage responses.

Some of these proliferating cells differentiate into cytotoxic T-cells (T_{c}) which destroy cells having the selected antigen. After the antigen is no longer present, the mature clonally selected cells will remain as memory helper and memory cytotoxic T-cells, which will circulate in the body and recognize the antigen should it show up again. If the antigen triggering this response is not a foreign antigen, but a self antigen, the result is autoimmune disease; if the antigen is an antigen from transplanted tissue, the result is graft rejection.

The CD4 glycoprotein is a receptor expressed on the surface of a T-cell subset and macrophages. In general, CD4+ T-cells function as Tₕ cells. The CD4 receptor participates in the antigen MHC class II recognition of T-cells.

Recent studies have demonstrated the importance to the immune system of the CD40 ligand (CD40L, also known as CD154, gp39, T-BAM and TRAP), a glycoprotein expressed primarily on activated CD4+ T cells, and the CD40 receptor, which is expressed on a variety of APCs. Grewal et al., Immunological Research, 16, 59 (1997), disclose that CD40L/CD40 interactions are involved in the humoral immune response, as well as cell-mediated immune responses and T-cell-mediated effector functions that are required for proper functioning of the host defense system.

A critical issue in transplant immunology is to determine how the components and regulatory interactions involved in graft rejection might be manipulated to allow graft acceptance. One form of immunosuppressive therapy used clinically and experimentally is that achieved by the administration of isolated, purified antibody preparations. Therapeutic antibodies act in one of two ways. Lytic antibodies, also referred to as depleting antibodies, kill lymphocytes in vivo by targeting them for destruction. Nonlytic antibodies, or nondepleting antibodies, act by blocking the function of the target antigen without killing the cell that bears it.

Recently, monoclonal antibodies (mAbs) such as OKT3, a mouse antibody directed against the CD3 antigen of humans, have become widely used in clinical transplantation settings. However, the interaction of OKT3 with the CD3 antigen initially activates T cells, which stimulates the release of lymphokines, leading to significant clinical side effects.

The use of non-depleting anti-CD4 mAbs has been disclosed to inhibit a number of allograft rejections, including allogeneic cutaneous, renal, and cardiac tissue transplants. See, e.g., U.S. Pat. No. 5,690,933; WO 96/36359; Onodera et al., Transplantation 68, 288 (1996); and Lehmann et al., Transplantation, 64, 1181 (1997).

The role of anti-CD40L antibodies, either alone or in combination with other immunosuppressive agents, has been studied in allo- and/or xenografts. See, e.g., WO 98/52606; WO 98/59669; Harlan and Kirk, Graft, 1, 63 (1998); and Kenyon et al., Proc. Natl. Acad. Sci., U.S.A., 96, 8132 (1999). Parker et al., Proc. Natl. Acad. Sci., U.S.A., 92, 9560 (1995), disclosed that the infusion of allogeneic small lymphocytes prior to transplant in combination with the use of an anti-CD40L antibody led to a more than 100 day pancreatic islet allograft survival in a mouse model. Larsen et al., Nature, 381, 434 (1996), disclosed that the use of a combination of an anti-CD40L antibody and an anti-CD28 antibody delayed the rejection of skin allografts beyond 50 days. However, when an anti-CD4 antibody was used alone or added to the anti-CD40L and anti-CD28 combination, Larsen et al. disclosed that the allografts were rejected with mean survival time (MST) of less than 20 days. Thus, it remains unclear whether these antibodies will be effective clinically and under what circumstances.

If clinically applicable anti-rejection antibody regimens could be developed, then the transplantation of xenogeneic islets could become an effective means for treating or even curing patients with diabetes. Therefore, a need exists for compositions and methods to increase the applicability of islet transplantation for the treatment of diabetes.

### SUMMARY OF THE INVENTION

The present invention provides a method for in vivo immunosuppression in humans and mammals. The method includes pretreatment and post-transplant in vivo therapy to inhibit or prevent the rejection of transplanted islet cells. Preferably, the present method can impart durable tolerance to the recipient, rather than just delay the rejection of the implanted cells. The present invention also provides a method to treat autoimmune disorders and diseases.

Specifically, the method of the present invention comprises administering to a mammal, such as a human, in need of such treatment an effective immunosuppressive amount of a combination of at least one compound which binds specifically to a CD40 ligand present on T-cells so as to interrupt binding to a CD40 receptor, and at least one compound which binds specifically to a CD4 receptor present on T-cells so as to interrupt binding with an antigen-MHC complex, such as a non-depleting anti-CD4 antibody.

The term “antibody”, as used herein, includes human and animal mAbs, and preparations of polyclonal antibodies, as well as antibody fragments, synthetic antibodies, including recombinant antibodies, chimeric antibodies, including partially and fully humanized antibodies, anti-idiotopic antibodies and derivatives thereof.

The term “compound” is meant to indicate, for example, antibodies as defined herein, and molecules having antibody-like function, such as synthetic analogues of antibodies, e.g., single-chain antigen binding molecules, small binding peptides, or mixtures thereof.

Preferably, the compounds of the present method are antibodies. More preferably, one of the antibodies administered in the combination will be capable of specifically binding to the CD40 ligand, and one of the antibodies administered in the combination will be capable of specifically binding to the CD4 receptor.

The term “islet cell” includes any mammalian organ, tissue or cell, capable of producing insulin in vivo, including synthetic or semi-synthetic cells, or transgenic cells.

As mentioned hereinabove, the method of the present invention is useful in the treatment of islet cell transplant rejection. More specifically, the method may be employed for the treatment of a patient that has undergone islet cell transplantation that is allogeneic or xenogeneic. In one embodiment of the invention, the mammalian recipient is xenogeneic to transplanted porcine islets. In another embodiment of the invention, the mammalian recipient is allogeneic to transplanted porcine islets. Furthermore, the method of the present invention may be utilized prior to, following or concurrently with the transplant procedure, or any combination thereof.

In a further embodiment of the method of the present invention, an anti-inflammatory or immunosuppressive drug may be administered prior to, following, or concurrently with the combination of compounds described hereinabove. For example, suitable drugs for this purpose include, but are not limited to, cyclosporin, FK506, rapamycin, corticosteroids, cyclophosphamide, mycophenolate mofetil, leflunomide, deoxyspergualin, azathioprine, OKT-3 and the like.

As used herein, the term “immune tolerance” or simply “tolerance” is intended to refer to the durable active state of unresponsiveness by lymphoid cells to a preselected or specific antigen or set of antigens. The immune response to other immunogens is thus unaffected, while the requirement for sustained exogenous immunotherapy can be either reduced or is eliminated. Additionally, tolerance enables subsequent transplantation of material comprising the same antigen or set of antigens without increasing the need for exogenous immunotherapy.

As used herein, the term “treating”, with respect to an autoimmune disease or condition, includes preventing or delaying the onset or flare-up of the disease or condition, as well as reducing or eliminating one or more symptoms of the disease or condition, such as inflammation, fever and the like, after onset.

### BRIEF DESCRIPTION OF THE DRAWINGS

FIG. 1 depicts graphically data showing the level of plasma glucose over time in animals treated with anti-CD40L antibody and anti-CD4 antibody combination therapy following transplant.

FIG. 2 graphically depicts results of IVGTT analyses for normal Lewis rats, at an early time point (day 50) and prior graft nephrectomy.

FIG. 3A presents graphically data showing the level of rat insulin from insulin extraction tests.

FIG. 3B presents graphically data showing the level of rat C-peptide.

### DETAILED DESCRIPTION OF THE INVENTION

T cell activation, and immunological processes dependent thereon, requires both T cell receptor (TCR) mediated signals and simultaneously delivered costimulatory signals. An important costimulatory signal is delivered by the ligation of CD40 on an antigen-presenting cell, such as a B cell, by CD40L on a T cell. CD40 has been molecularly cloned and characterized. Stamenkovic et al., EMBO J., 8, 1403 (1989). Human CD40 is a 50 kD cell surface protein expressed on mature B cells, as well as on macrophages and activated endothelial cells. CD40 belongs to a class of receptors involved in programmed cell death, including Fas/CD95 and the tumor necrosis factor (TNF) alpha receptor.

CD40L has also been molecularly cloned and characterized. Armitage et al., Nature, 357, 80 (1992); Lederman et al., J. Exp. Med., 175, 1091 (1992); and Hollenbaugh et al., EMBO J., 11, 4313 (1992). Human CD40L is a 32 kD type II membrane glycoprotein with homology to TNF alpha that is transiently expressed, primarily on activated T cells. Binding between the CD40L and its receptor, CD40L, has been shown to be required for all T cell-dependent antibody responses. In particular, CD40:CD40L binding provides anti-apoptotic and/or lymphokine stimulatory signals.

The importance of CD40:CD40L binding in promoting T cell dependent biological responses was more fully appreciated when it was discovered that X-linked hyper-IgM syndrome (X-HIGM) in humans is the phenotype resulting from genetic lack of functional CD40L. Affected individuals have normal or high IgM levels, but fail to produce IgG, IgA or IgE antibodies, and suffer from recurrent, sometimes severe, bacterial and parasitic infections, as well as an increased incidence of lymphomas and abdominal cancers. A similar phenotype is observed in non-human animals rendered nullizygous for the gene encoding CD40L (knockout animals). B cells of CD40L nullizygotes can produce IgM in the absence of CD40:CD40L binding, but are unable to undergo isotype switching, or to survive normally after affinity maturation. Histologically, lymph node germinal centers fail to develop properly, and memory B cells are absent or poorly developed. Functionally, these defects contribute to a severe reduction or absence of a secondary (mature) antibody response. Defects in cellular immunity are also observed, manifested by an increased incidence of bacterial and parasitic infections. Many of these cell-mediated defects are reversible by administration of IL-12 or IFN-gamma. These observations substantiate the view that normal CD40:CD40L binding promotes the development of Type I T-helper cell immunological responses.

A number of preclinical studies have established that agents capable of interrupting CD40:CD40L binding have promise as immunomodulating agents. In particular, studies involving small-animal organ or tissue transplantation models have shown that CD40:CD40L interruptors promote survival of allogeneic grafts. In selected models, transient administration of agents interfering with T cell costimulation has resulted in the induction of indefinite graft acceptance. Interruption of CD40:CD40L binding in particular has yielded promising results, since it appears that engagement of this counter-receptor pair precedes other costimulatory signals in chronology and hierarchy. Ranheim et al., J. Exp. Med., 177, 925 (1993); Roy et al., Eur. J. Immunol., 25, 596 (1995); Han et al., J. Immunol., 155, 556 (1995); Shinde et al., J. Immunol., 157, 2764 (1996), Yang et al., Science, 273, 1862 (1996); Grewal et al., Science, 273, 1864 (1996); and Lederman et al., J. Immunol., 149, 3817 (1992). Blockade of CD40:CD40L binding has resulted in prolongation of cardiac (Larsen et al., Transplantation, 61, 4 (1996); Larsen et al., Nature, 381, 434 (1996)), cutaneous (Larsen et al., Nature, 381, 434 (1996); Markees et al., Transplantation, 64, 329 (1997)) and islet allografts (Parker et al., Proc. Natl. Acad. Sci. USA, 92, 9560 (1995); Rossini et al., Cell Transplant, 5, 49) in rodents, and of allogeneic kidneys in primates (Kirk et al., Proc. Natl. Acad. Sci. USA, 194, 8789 (1997)). It has also been demonstrated to delay onset of autoimmune diabetes in non-obese diabetic (NOD) mice (Balasa et al., J. Immunol., 159, 4620 (1997)). Lastly, it has been reported that interference with CD40:CD40L binding prevents the production of inflammatory cytokines (Dechanet et al., J. Immunol., 159, 5640 (1997); Kiener et al., J. Immunol., 155, 4917 (1995)).

CD40:CD40L blockade thus may provide potentially powerful therapies for prevention of islet allograft or xenograft failures in individuals having defective glucose metabolism, such as Type I diabetes. However, as noted above, studies in rodent model systems have correlated poorly with the outcome of testing or therapy of large animals, including primates and humans.

Disclosed herein are studies assessing the effects of a preferred combination of a CD40L blocking agent, a humanized mAb having the antigen-specific binding properties of mAb 5c8 (Lederman et al., J. Exp. Med., 175, 1091 (1992)), and a CD4 receptor blocking agent, such as RIB 5/2 (Lehmann et al., Transplantation, 54, 959 (1992)), in animal models of xenogeneic islet cell transplantation.

The following discussion illustrates and exemplifies the variety of contexts and circumstances in which the invention can be practiced, as well as providing proof-of-principle studies involving specific embodiments of the invention.

Recipient Hosts

The invention can be used for treatment or prophylaxis of any mammalian recipient of an islet cell graft, or any mammal in need of an islet cell graft. Recipient hosts (also referred to as recipients or hosts) accordingly are afflicted with, or at risk of, a defect in metabolic control of blood glucose metabolism (glucose homeostasis). For example, the recipient can be hyper- or hypo-glycemic. The invention is particularly suitable for use with diabetic recipients, particularly recipients afflicted with diabetes mellitus (DM). Preferably, the recipient is a primate, more preferably a higher primate, most preferably a human. In other embodiments, the recipient may be another mammal in need of a tissue graft, particularly a mammal of commercial importance, or a companion animal or other animal of value, such as a transgenic animal, cloned animal, or a member of an endangered species. Thus, recipient hosts also include, but are not limited to, sheep, horses, cattle, goats, pigs, dogs, cats, rabbits, guinea pigs, hamsters, gerbils, rats and mice.

Donor or Graft Tissue

The invention can be used with any type of insulin-producing tissue transplant or graft procedure, particularly procedures wherein the donor (graft) tissue is affected by, or at risk of, failure or rejection by the recipient host's immune system. In particular, the invention can be used in any context wherein the donor tissue is not histocompatible (MHC-compatible) with the recipient host. Thus, in addition to autologous or syngeneic donor tissue, the invention can be used with allogeneic or xenogeneic donor tissue. The donor tissue can be derived, by conventional means, from a volunteer or other living donor, or from a cadaveric donor. In one embodiment, the donor is as histocompatible as practicable with the recipient host. For example, where the recipient host is a human, autologous and allogeneic donor tissue is used. In another embodiment, the donor tissue can be obtained from a heterologous species (in which case it is referred to as a heterograft), such as a non-human primate, e.g., a chimpanzee or a baboon, or a member of the porcine species, e.g., a pig.

In some embodiments, the donor islet cells comprise a part, portion or biopsy of a donor pancreas which comprises insulin-producing cells. If a cadaveric donor is used, the pancreas is preferably exposed to cold ischemic conditions for no more than about eight hours. In still other embodiments, the donor islet cells comprise isolated or suspended islets or islet cells, including cells withdrawn or excised from a fetal or adult donor, cells maintained in primary culture, or an immortalized cell line. Appropriate means for preparing donor islets or islet cell suspensions from whole pancreata are well known (see, e.g., Ricordi et al., Diabetes, 37, 413 (1988); Tzakis et al., Lancet, 336, 402 (1990); Linetsky et al., Diabetes, 46, 1120 (1997)). Appropriate pancreata are obtained from donors essentially free of defects in blood glucose homeostasis. Other sources of insulin-producing cells include islet progenitor cells, such as fetal cells, optionally expanded in primary culture. Any appropriate cell type can be used, however, including cells harboring exogenous genetic material encoding an expressible insulin gene. Thus, the invention encompasses the use of transfected or transformed host cells, which have been (or are derived from ancestor cells which have been) engineered to express insulin, either constitutively or inducibly (e.g., under control of a glucose-responsive promoter or enhancer). In other embodiments, the invention encompasses the use of pancreatic or other donor cell types derived from a transgenic mammal that has been engineered to include genetic material necessary for the production of insulin in some or all of its body tissues.

The insulin producing tissue (donor tissue) is introduced systemically or locally into the recipient host. For example, isolated, suspended or dispersed insulin-producing cells can be infused intravascularly, or implanted into a desired site, such as a bone marrow cavity, the liver, within the kidney capsule, intramuscularly, or intraperitoneally. In some embodiments, the cells are mitotically competent and produce new tissue of donor origin. In other embodiments, the cells are not mitotically competent, but remain viable in the donor, and produce or express insulin. In any event, an effective amount of insulin-producing cells or tissue is implanted, by which is meant an amount sufficient to attenuate (detectably mitigate) the recipient's defect in glucose metabolism (e.g., hypoglycemia or hyperglycemia). Optimally, the amount is sufficient to restore the recipient's ability to maintain glucose homeostasis, so as to free the recipient from dependence on conventional (e.g., injected or inhaled) insulin replacement therapy.

In some embodiments, the insulin-producing tissue is physically separated (isolated) from surrounding tissues of the recipient by an immunoisolation device. Appropriate devices protect the insulin-producing tissue from most effectors of cellular and humoral immunity, including but not limited to, leukocytes, immunoglobulin and complement. Thus, the immunoisolation device generally provides a semipermeable barrier, such as a membrane, having a pore size sufficient to prevent diffusion therethrough of molecules more massive than about 50 to 100 kD. The barrier defines an isolation chamber in which the insulin-producing tissue is disposed, and is free of any sites at which the insulin-producing tissue can physically contact cells or tissues external to the barrier. Any conventional device, envelope, capsule or microcapsule can be used, including single- or double-walled alginate microcapsules (e.g., as described in U.S. Pat. No. 5,227,298). Other conventional microcapsules include alginate polylysine microcapsules, chemically cross-linked alginate microcapsules, and capsules formed of other biocompatible polymers, formed into a structurally sound immunoisolation device of any desired shape or size (see, e.g., Jaink et al., Transplantation, 61, 4 (1996)).

Exemplary CD4 Receptor Binding Interruptors

CD4 receptor blocking agents useful for practice of the invention include any compound that blocks the interaction of cell surface CD4 (e.g., expressed on Tₕ cells) with an antigen-MHC complex. Compounds that are specifically contemplated include polyclonal antibodies and monoclonal antibodies (mAbs), as well as antibody derivatives such as chimeric molecules, humanized molecules, molecules with reduced effector functions, bispecific molecules, and conjugates of antibodies.

Monoclonal antibodies against the murine CD4 (L3T4) antigen have been disclosed as immunosuppresive agents for the control of humoral immunity, transplant rejection and autoimmunity. See, e.g., Siegling et al., Transplantation 5, 464 (1994); and U.S. Pat. No. 5,690,933. In addition, CD4 mAbs have been shown to create a tolerance-permissive environment in vivo, which can achieve tolerance to certain soluble protein antigens as well as transplantation antigens. However, the mechanism(s) by which CD4 mAbs produce these effects are not clear. In most previous reports, immunosuppression was obtained under conditions that depleted target cells in vivo. A simple interpretation was that the immune suppression so achieved was due to the absence of CD4 T cells. A depleting antibody is an antibody which can deplete more than 50%, for example, from 90 to 99%, of target cells in vivo. Depleting anti-CD4 monoclonal antibodies reported in the literature include L3T4 and BWH-4. See, e.g., Takeuchi et al., Transplantation, 53, 1281 (1992); and Sayegh et al., Transplantation, 51, 296 (1991).

On the other hand, in vitro experiments have demonstrated that CD4 mAbs can affect lymphocyte functions simply through binding to the antigen on the cell surface, without causing cell lysis. In addition, immunosuppression and tolerance induction has been obtained in vivo with the use of sublytic concentrations of CD4 mAbs, and by F(ab′)₂ CD4 mAb fragments, which suggests that for mAb-mediated immune regulation the depletion of target cells may not be essential. The use of nondepleting CD4 antibodies has been disclosed to produce tolerance to foreign immunoglobulins, bone marrow and skin grafts. See, e.g., U.S. Pat. No. 5,690,933.

Lehmann et al., Transplantation, 54, 959 (1992) previously described the non-depleting anti-CD4 mAb RIB 5/2. This publication discloses the use of RIB 5/2 to prevent the rejection of rat skin allografts. Furthermore, Siegling et al., Transplantation, 57, 464 (1994), disclose that RIB 5/2 monotherapy induces survival of renal allografts in a rat model; Lehmann et al., Transplantation, 64, 1181 and Onodera et al., Transplantation, 68, 288 (1999), disclose the immune effects of RIB 5/2 monotherapy in allograft models; and Onodera et al., The Journal of Immunology, 157, 1944 (1996) disclose that treatment with RIB 5/2 abrogated the rejection of cardiac allografts in sensitized rat recipients. However, these publications do not disclose the use of any anti-CD4 blocking agent for the treatment or prevention of xenogeneic transplant rejection.

U.S. Pat. No. 5,690,933 disclosed a hybridoma which produces a non-depleting anti-CD4 monoclonal antibody known as YTS 177.9 (deposited at the European Collection of Animal Cell Cultures, Porton Down, G.B., under ECACC Accession No. 90053005). In addition, PCT application WO 96/36359 discloses a non-depleting CD4 antibody, specifically, a cdr-grafted anti-CD4 antibody designated OKT cdr4a.

Such antibodies can have the antigen-specific binding characteristics of the mAb RIB 5/2, as described in Lehmann et al., Transplantation, 54, 959 (1992). In one embodiment of this invention, the monoclonal antibody binds to the protein which the mAb RIB 5/2 binds.

Exemplary CD40:CD40L Binding Interruptors

Therapeutic compounds useful for practice of the invention include any compound that blocks the interaction cell surface CD40 (e.g., on B cells) with CD40L in situ, e.g., on the surface of activated T cells. CD40:CD40L binding interruptor compounds, such as CD40L blocking agents, include polyclonal antibodies and monoclonal antibodies (mAbs), as well as antibody derivatives such as chimeric molecules, humanized molecules, molecules with reduced effector functions, bispecific molecules, and conjugates of antibodies.

The CD40L-specific mAb MR1 (ATCC Accession No. HB 11048, as described in U.S. Pat. No. 5,683,693) has shown dramatic in vivo effects in mouse models of pancreatic islet allotransplantation. Parker et al., Proc. Natl. Acad. Sci., U.S.A., 92, 9560 (1995). Recently, selective inhibition of T-cell costimulation by the human homologue to MR1, the CD40L-specific mAb 5c8 (ATCC Accession No. HB 10916, as described in U.S. Pat. No. 5,474,771) significantly prolonged the survival of MHC-mismatched renal and islet allograft in non-human primates without the need for chronic immunosuppression.

In a preferred embodiment, the antibody has the antigen-specific binding characteristics of mAb 5c8. In one embodiment of this invention, the monoclonal antibody binds to the protein to which the mAb 5c8 binds. In another embodiment of this invention, the mAb binds to the epitope to which the mAb 5c8 binds. One preferred antibody for use in the present method is the humanized mAb 5c8. Other known antibodies against CD40L include antibodies ImxM90, ImxM91 and ImxM92 (obtained from Immunex), an anti-CD40L mAb commercially available from Ancell (clone 24-31, catalog #353-020, Bayport, Minn.), and an anti-CD40L mAb commercially available from Genzyme (Cambridge, Mass., catalog #80-3703-01). Also commercially available is an anti-CD40L mAb from PharMingen (San Diego, catalog #33580D). Numerous additional anti-CD40L antibodies have been produced and characterized (see, e.g., Bristol-Myers Squibb, PCT application WO 96/23071).

The invention also includes use of other CD40L blocking agents, such as complete Fab fragments, F(ab′)₂ compounds, V_{H} regions, F_{V} regions, single chain antibodies (see, e.g., PCT application WO 96/23071), polypeptides, fusion constructs of polypeptides, fusions of CD40 (such as CD40Ig, as in Hollenbaugh et al., J. Immunol. Meth. 188, 1 (1995)), and small molecules such as small semi-peptides or non-peptide agents, all capable of blocking or interrupting CD40:CD40L binding. Procedures for designing, screening and optimizing small molecules are provided in PCT/US96/10664, filed Jun. 21, 1996.

Monoclonal Antibodies

Monoclonal antibodies against the CD40L and/or CD4 receptor can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages over those produced by inoculation of animals, as they are highly specific, sensitive and relatively “pure” immunochemically. Immunologically active fragments of the present antibodies are also within the scope of the present invention, e.g., the F(ab) fragment, as are partially and fully humanized monoclonal antibodies.

The present invention includes a monoclonal antibody that is conjugated to a detectable label, for example, a radioisotope, fluorescent label or binding site for a detectable label.

It will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be provided by the techniques of recombinant DNA technology to yield derivative antibodies, humanized or chimeric molecules or antibody fragments which retain at least the specificity of the reference monoclonal antibody.

Recombinant Antibodies

Various forms of antibodies also can be produced using standard recombinant DNA techniques (Winter and Milstein, Nature, 349, 293 (1991)). Obviously, once one has an immortalized cell line, e.g., a hybridoma, or an RGDP containing DNA encoding at least a polypeptide component of a binding ligand, one skilled in the art is in a position to obtain (according to techniques well known in the art, see European patent application EPA 449,769) the entire nucleotide sequence encoding the ligand, e.g., the mAb secreted by the cell. Therefore, the present invention also encompasses primary nucleotide sequences which encode the ligands, e.g., mAbs as defined above, together with fragments of these primary sequences and secondary nucleotide sequences comprising derivatives, mutations and hybridizing partners of said primary nucleotide sequences.

These nucleotide sequences may be used in a recombinant system to produce an expression product according to standard techniques. Therefore, the present invention includes vectors (cloning and expression vectors) incorporating said nucleotide sequences, transformed cells incorporating said vectors and expression products produced by use of a recombinant system utilizing any such vectors or transformed cells.

Yet another possibility would be to produce a mutation in the DNA encoding the monoclonal antibody, so as to alter certain of its characteristics without changing its essential specificity. This can be done by site-directed mutagenesis or other techniques known in the art.

The production of fusion proteins is also contemplated. See, for instance, Stamenkovic et al, “The B Lymphocyte Adhesion Molecule CD22 Interacts with Leukocyte Common Antigen CD45RO on T Cells and α2-6 Sialytransferase, CD75, on B Cells,” Cell, 66, 1133 (1991).

The present invention also includes methods for expressing a ligand, e.g., a mAb, derivative, functional equivalent or fragment thereof, which comprises using a nucleotide sequence, vector or transformed cell as defined above.

In addition, standard recombinant DNA techniques can be used to alter the binding affinities of recombinant antibodies with their antigens by altering amino acid residues in the vicinity of the antigen binding sites. For example, the antigen binding affinity of an antibody may be increased by mutagenesis based on molecular modeling (Queen et al., Proc. Natl. Acad. Sci., 86, 10029 (1989); PCT application WO 94/04679). It may be desirable to increase or to decrease the affinity of the antibodies, depending on the targeted tissue type or the particular treatment schedule envisioned. This may be done utilizing phage display technology (see, e.g., Winter et al., Ann. Rev. Immunol., 12, 433 (1994); and Schier et al., J. Mol. Biol., 255, 28 (1996)). As an example, it may be advantageous to treat a patient with constant levels of antibodies with reduced affinity for CD40L for semi-prophylactic treatments. Likewise, antibodies with increased affinity for CD40L may be advantageous for short-term treatments.

Chimeric and Reshaped Antibodies

Published European patent EP 120694 (Boss et al/Celltech) describes the cloning and expression of chimeric antibodies. In these derivatives, the variable domains from one immunoglobulin are fused to constant domains from another immunoglobulin. Usually, the variable domains are derived from an immunoglobulin gene from one species, i.e., an animal species, e.g., a mouse or a rat, and the constant domains are derived from an immunoglobulin gene from a different species, perhaps a human. A later European patent application, EP 125023 (Cabilly/Genetech), and U.S. Pat. No. 4,816,567, describe the production of other variations of immunoglobulin-type molecules using recombinant DNA technology. Chimeric antibodies reduce the immunogenic responses elicited by animal antibodies when used for human therapy or prophylaxis.

Chimeric antibodies are constructed, for example, by linking the antigen binding domain from a mouse antibody to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobin light chain or heavy chain) (see, e.g., U.S. Pat. No. 4,816,567; Morrison et al., Proc. Natl. Acad. Sci., 81, 6851 (1984)).

Another possibility is to attach just the variable region of the monoclonal antibody to another non-immunoglobulin molecule, to produce a derivative chimeric molecule (see PCT application WO 86/01533, Neuberger and Rabbits/Celltech). A further possibility would be to produce a chimeric immunoglobulin having different specificities in its different variable regions, e.g., the monoclonal antibodies of the present invention (see European patent EP 68763).

European patent EP 239400 (Winter) describes how it is possible to make an altered, derivative, antibody by replacing the complementarity determining regions (CDRs) of the variable domain of an immunoglobulin with the CDRs from an immunoglobulin of different specificity, using recombinant DNA techniques—so called “CDR-grafting”. This enables altering the antigen-binding specificity of an antibody. (In the present case it might be the CDRs of RIB 5/2, of 5c8, of an antibody with the same binding specificity as these anti-CD4 and anti-CD40L antibodies, or of antibodies which is cross-reactive with RIB 5/2 or 5c8 which are transferred to another antibody.) Thus, CDR grafting enables “humanization” of antibodies, in combination with alteration of the variable domain framework regions.

Humanized antibodies are antibodies initially derived from a nonhuman mammal in which recombinant DNA technology has been used to substitute some or all of the amino acids not required for antigen binding with amino acids from corresponding regions of a human immunoglobin light or heavy chain. That is, they are chimeras comprising mostly human immunoglobulin sequences into which the regions responsible for specific antigen-binding have been inserted (see, e.g., PCT patent application WO 94/04679).

Humanized antibodies minimize the use of heterologous (inter-species) sequences in antibodies for use in human therapies, and are less likely to elicit unwanted immune responses. For example, a “humanized” antibody containing the CDRs of a rodent antibody specific for an antigen of interest might well be less likely to be recognized as foreign by the immune system of a human. It follows that a “humanized” antibody with the same binding specificity as, e.g., mAb RIB 5/2, mAb 5c8, or an antibody that cross-reacts with either might well be of particular use in human therapy and/or diagnostic methods.

A humanized antibody may be produced, for example, animals may be immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Primatized antibodies can be produced similarly.

Another embodiment of the invention includes the use of human antibodies, which can be produced in nonhuman animals, such as transgenic animals harboring one or more human immunoglobulin transgenes. Such animals may be used as a source for splenocytes for producing hybridomas, as described in U.S. Pat. No. 5,569,825. Human antibodies can also be directly provided by reconstituting the human immune system in mice lacking their native immune system, then producing human antibodies in these “humanized mice.”

Antibody fragments and univalent antibodies also can be used in practice of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab′)₂), whether any of the above are covalently or non-covalently aggregated, so long as the aggregation is capable of selectively reacting with a particular antigen or antigen family.

Anti-Idiotopic Antibodies

The provision of an antibody such as RIB 5/2 or 5c8 allows persons skilled in the art to obtain binding partners, e.g., antigens/epitopes or antibody/paratopes which bind to it. Therefore, the present invention also provides binding partners, e.g., antigens and/or antibodies which bind with an antibody or derivatives thereof as hereby provided, such as RIB 5/2 and 5c8.

The binding partners obtained by use of the RIB 5/2 mAb and 5c8 mAb may also be used to produce additional ligands, e.g., antibodies other than RIB 5/2 or 5c8 (or molecules having antibody-like binding function, e.g., fragments, derivatives and synthetic analogues of antibodies such as single-chain antigen-binding molecules). Therefore, also provided are ligands, e.g., mAbs which are able to bind with a binding partner which is able to bind with the RIB 5/2 mAb and 5c8 mAb. Such ligands (“cross-reactive ligands”), e.g., mAbs may recognize the same epitope as recognized by RIB 5/2 mAb and 5c8 mAb on said binding partner.

The present invention also provides derivatives, functional equivalents (e.g., a molecule having an antibody-like binding specificity) and fragments of said cross-reactive ligands, perhaps produced using one or more of the techniques of recombinant DNA technology referred to and discussed above. Also included are single domain ligands (mAbs) as described in PCT application WO 90/05144.

Antigen Isolation

Using standard techniques, it is possible to use a ligand, e.g., antibodies of the present invention and derivatives thereof, in the immunopurification of a binding partner antigen. Techniques for immunoaffinity column purification are well known, see for instance “Current Protocols in Immunology,” ed. J. E. Coligan et al, John Wyley and Sons, Unit 8.2. Isolation of the epitope and compounds binding to the epitope are contemplated by this invention. For example, the mAb RIB 5/2, which is directed against the CD4 receptor, will bind to a CD4 epitope on CD4+ cells. Similarly, mAb 5c8 will bind to an epitope on CD40L in cells expressing CD40L. These epitopes may then be purified, for instance utilizing an immunoaffinity column (as discussed), and partially or wholly sequenced, for instance using repeated rounds of Edman degradation.

In addition, it should be possible to use an immunoaffinity column to isolate cross-reactive ligands as discussed above, without needing to isolate the antigens themselves. A first round of immunoaffinity purification uses a ligand, e.g., mAb RIB 5/2, mAb 5c8, etc., to remove from a sample the antigen-containing binding partner, which may then be used in the column to select, from a heterogeneous population of ligands, those ligands which are cross-reactive with mAb RIB 5/2, mAb 5c8, etc., and recognize the same binding partners.

A binding partner, such as a peptide or small binding molecule, isolated using the ligand, e.g., mAb RIB 5/2, mAb 5c8, etc., may be used to select cross-reactive ligands from a repertoire or heterogenous population of antibodies generated by a wide variety of means. One way is to select monoclonal antibodies and cell lines producing them by the standard hybridoma techniques. Also provided by the present invention are immortalized cells, e.g., hybridomas producing said cross-reactive ligands.

Another way of selecting ligands which are cross-reactive with a ligand such as the RIB 5/2 mAb or 5c8 mAb is to use the methods for producing members of specific binding pairs disclosed in PCT application WO 92/01047 (Cambridge Antibody Technology Limited and MRC/McCafferty et al.). This publication discloses expression of polypeptide chain components of a genetically diverse population of specific binding pair members, such as antibodies, fused to a component of a secreted replicable genetic display package (RGDP), such as a bacteriophage, which thereby displays the polypeptide on the surface. Very large repertoires of displayed antibodies may be generated, and screened by means of antigen binding to obtain one or more antibodies of interest, along with the DNA encoding them. DNA encoding for a polypeptide displayed on the surface of an RGDP is contained within the RGDP and may therefore be easily isolated and cloned for expression. The antibody repertoire screen may of course be derived from a human source.

Routes of Administration

The CD40L and CD4 binding interrupters, such as an anti-CD40L antibody and an anti-CD4 antibody, used in the invention can be administered in any manner which is medically acceptable. Depending on the specific circumstances, local or systemic administration may be desirable. Preferably, the agent is administered via a parenteral route such as by an intravenous, intraarterial, subcutaneous, intramuscular, intraorbital, intraventricular, intraperitoneal, subcapsular, intracranial, intraspinal, or intranasal injection, infusion or inhalation. The agent also can be administered by implantation of an infusion pump, or a biocompatible or bioerodable sustained release implant, into the recipient host, either before or after implantation of donor tissue. Alternatively, certain compounds of the invention, or formulations thereof, may be appropriate for oral or enteral administration. Still other compounds of the invention will be suitable for topical administration.

In further embodiments, the CD40L and CD4 antibodies are provided indirectly to the recipient, by administration of a vector or other expressible genetic material encoding the antibodies. The genetic material is internalized and expressed in cells or tissue of the recipient, thereby producing the interruptor in situ. For example, a suitable nucleic acid construct would comprise sequence encoding one or more of the mAb 5c8 immunoglobulin (Ig) chains (as disclosed in U.S. Pat. No. 5,474,771) and/or one or more of the mAb RIB 5/2 Ig chains. Other suitable constructs would comprise sequences encoding chimeric or humanized versions of the mAb 5c8 Ig chains or antigen-binding fragments thereof, and/or mAb RIB 5/2 Ig chains or antigen-binding fragments thereof. Still other suitable constructs would comprise sequences encoding part or all of other CD40L-specific mAbs and/or CD4-specific mAbs. The construct is delivered systemically or locally, e.g., to a site vicinal to the site of implantation of insulin-expressing tissue.

Alternatively, the vector or other genetic material encoding the CD40L antibody and/or CD4 antibody is internalized within a suitable population of isolated cells to produce interruptor-producing host cells. These host cells then are implanted or infused into the recipient, either locally or systemically, to provide in situ production of the CD40L antibody and/or CD4 antibody. Appropriate host cells include cultured cells, such as immortalized cells, as well as cells obtained from the recipient (e.g., peripheral blood or lymph node cells, such as natural killer (NK) cells).

In general, the active agents of the invention are administered to the recipient host. However, the compounds also can be administered to the donor, or to the donor tissue. For example, an antibody or antibodies of the present invention can be included in a perfusion or preservative fluid in which the donor tissue is stored or transported prior to its integration into the recipient host.

Formulation

In general, the agents used in practice of the invention are suspended, dissolved or dispersed in a pharmaceutically acceptable carrier or excipient. The resulting therapeutic composition does not adversely affect the recipient's homeostasis, particularly electrolyte balance. Thus, an exemplary carrier comprises normal physiologic saline (0.15M NaCl, pH 7.0 to 7.4). Other acceptable carriers are well known in the art and are described, for example, in Remington's Pharmaceutical Sciences, Gennaro, ed., Mack Publishing Co., 1990. Acceptable carriers can include biocompatible, inert or bioabsorbable salts, buffering agents, oligo- or polysaccharides, polymers, viscosity-improving agents, preservatives, and the like.

Any CD40L binding interruptor or CD4 binding interruptor, such as an anti-CD40L antibody or an anti-CD4 antibody, that is used in practice of the invention is formulated to deliver a pharmaceutically-effective or therapeutically-effective amount or dose, which is an amount sufficient to produce a detectable, preferably medically beneficial effect on the recipient. Medically beneficial effects would include preventing, delaying or attenuating deterioration of, or detectably improving, the recipient's medical condition. As an example, renal function and health of a kidney allograft or xenograft can be monitored by routinely measuring the concentrations of blood urea nitrogen or creatinine, or the volume or solute contents of urine, or the rate of clearance of relevant solutes from the blood into the urine. Similarly, glucoregulatory function and health of insulin-producing allograft or xenograft can be monitored by routinely measuring the concentrations of blood or urine glucose, glucose metabolites, or insulin, or measuring insulin response to glucose challenge, e.g., in a conventional glucose tolerance test.

Thus, an effective amount of a therapeutic agents of the invention, such as a CD40L antibody and a CD4 antibody, is any amount which detectably decreases the recipient's dependence on insulin replacement therapy. An optimal effective amount is one which substantially frees the recipient of dependence on exogenous insulin. More specifically, an effective amount is one which induces partial or substantially complete engraftment (acceptance and function) of donor insulin-producing tissue.

Dosages and Frequency of Treatment

The present invention provides a combination of one or more agents capable of binding to CD40L, and one or more agents capable of binding to CD4 for administration to patients who have received allografts and/or xenografts. The invention includes the use of the combination in an appropriate pharmaceutical formulation such as a unit dosage form, along with one or more drugs used to suppress rejection induced by pre-existing antibodies. Such drugs could include cyclophosphonamide, Deoxyspergualin and the like.

The amount of and frequency of dosing for any particular agent to be used in practice of the invention is within the skills and clinical judgement of ordinary practitioners of the tissue transplant arts, such as transplant surgeons. The general dosage and administration regime is established by preclinical and clinical trials, which involve extensive but routine studies to determine effective, e.g., optimal, administration parameters for the desired agent. Even after such recommendations are made, the practitioner will often vary these dosages for different recipient hosts based on a variety of considerations, such as the recipient's age, medical status, weight, sex, and concurrent treatment with other pharmaceuticals. Determining effective dosage and administration regime for each combination of CD40L antibody and CD4 antibody used to inhibit graft rejection is a routine matter for those of skill in the pharmaceutical and medical arts. The dosage amount and time course of should be sufficient to produce a clinically beneficial change in one or more indicia of the recipient's health status.

Appropriate dosages of any of said agents will, of course, vary, e.g., depending on the condition to be treated (for example the disease type or the nature of resistance), the effect desired, and the mode of administration. Dosages effective in humans can be derived from dosages effective in mice and other mammals by methods known to the art, i.e., U.S. Pat. No. 5,035,878.

In general, however, satisfactory results are obtained on administration parenterally, e.g., intravenously, for example by i.v. drip or infusion, at dosages of each agent on the order of from 0.01 to 2.5 up to 5 mg/kg, e.g., on the order of from 0.05 or 0.1 up to 1.0 mg/kg. Suitable dosages for human patients are thus on the order of from 0.5 to 125 up to 250 mg iv, e.g., on the order of from 2.5 to 50 mg i.v. The agents may be administered daily or every other day or less frequently at diminishing dosages to maintain a minimum level of agents in the blood during the antigen challenge, e.g., following organ transplant or during the acute phase of an autoimmune disease.

The pharmaceutical compositions of the present invention may be manufactured in conventional manner. A composition according to the invention is preferably provided in lyophilized form. For immediate administration it is dissolved in a suitable aqueous carrier, for example sterile water for injection or sterile buffered physiological saline. If it is considered desirable to make up a solution of larger volume for administration by infusion rather as a bolus injection, it is advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.50% by weight of the saline solution.

In clinical tests, for example, patients about to islet transplantation are selected for prophylactic therapy. On the day of transplantation, 2 hours prior to surgery, a first intravenous infusion of the CD40L antibody and/or the CD4 antibody is administered at a dose of 0.2 mg of each antibody per kg of body weight. Two days after surgery an identical infusion of the combination and/or individual antibody at 0.4 mg/kg of body weight is administered and then repeated at weekly intervals for one month. The intravenous infusions are prepared as follows: the lyophilized antibodies are mixed together and dispersed into 100 ml sterile buffered saline containing 4.51% by weight of human albumin. This saline dispersion is administered to the patients over a 30 minute period.

Adjuvant Agents

It is also contemplated that an anti-CD40L and anti-CD4 combination of the invention may be given alone or with standard immunosuppressant or anti-inflammatory agents. These would include cyclosporin, FK-506, Leflunomide, Rapamycin, cyclophosphamide, mycophenolate mofetil, Deoxyspergualin, corticosteroids, azathiorpine, OKT-3 and the like, and others. Use of the compounds and/or antibodies of the invention is expected to reduce the dosage requirements for such drugs and thereby to reduce undesired side effects. The compounds may also be used in combination with other monoclonal antibodies or other compounds specifically recognizing particular lymphocyte sub-populations, e.g., CD25 mAbs, CD45RB mAbs, CTLA4-Ig fusion peptide, etc.

Ex Vivo, Conditioning of Recipient's Lymphocytes

In some cases, immune suppression and/or tolerization may be enhanced by administering an amount of lymphocytes derived from the recipient that have been conditioned in vivo or ex vivo with the combination of anti-CD40L and anti-CD4 antibodies useful in the present invention. The conditioned or anergized lymphocytes can be given before, simultaneously with, or following transplantation and/or administration of the combination of antibodies, in an amount effective to induce or assist in inducing immune tolerance in the recipient. The lymphocytes preferably are obtained from the recipient prior to transplantation or other treatment, preconditioned by exposure to the antibodies employed in the present method, and exposed to the antigens on the donor material, prior to re-introduction into the recipient.

The invention will now be further described by reference to the following detailed examples.

### EXAMPLE 1

Evaluation of Immunosuppressive Drugs in the Prevention of Islet Cell Xenograft Rejection

Pancreata were removed from donor outbred female pigs, >2 years old, by standard surgical technique. Following removal, pancreata were perfused with Liberase HI (Roche Diagnostics Corp., Indianapolis, Ind., U.S.A., Cat. No. 1666720) for intraductal distension. Islet cells were dissociated from the perfused pancreata by the automated method. Cleaved islets were separated from non-islet tissue by continuous OptiPrep gradients (Accurate Chemical and Scientific Corp., Westbury, N.Y., U.S.A., Cat. No. AN-1030061) on a COBE 2991 cell separator (Gambro BCT International, Lakewood, Colo., U.S.A.). The resulting free floating, purified islet cells were cultured in M199 medium supplemented with 20% donor pig serum for 48 hours at 37° C.

Transplantation was conducted by standard surgical technique. Briefly, the recipient animals, non-diabetic inbred male Lewis rats weighing 250-270 grams, were anesthetized with Telazol 0.20 mg/kg BW, administered i.m. In addition, the analgesic buprinorphine was administered s.c. to the recipient animals. Transplantation was conducted by making a left flank incision on the recipient, into which 2,000 donor islet equivalents (IE) were injected under the left kidney capsule via PE-50 tubing according to standard procedure.

In this study, recipients were administered the following immunosuppressive drugs: anti-CD40L mAb AH.F5 (Biogen, Inc., Cambridge, Mass., U.S.A.; 12 mg/kg BW, administered i.p. on day—1, 0, 1, 7, and then twice weekly); non-depleting anti-CD4 mAb RIB 5/2 (20 mg/kg BW, administered i.p. on day—1, 0, 1, 2, 3, 5, and then twice weekly); Ha4/8, a non-specific control to anti-CD40L (12 mg/kg BW, administered i.p. on day—1, 0, 1, 7, and then twice weekly); and FK-506 (Prograf, Fujisawa, Inc.; diluted to 1.25 mg/ml in sterile water, 0.3 mg/kg BW, administered i.m. daily from day—2, i.e., 2 days prior to transplant).

Animals were sacrificed by intracardiac exsanguination after ether anesthesia on day +12 post transplant for histological analysis of the graft. Kidneys bearing xenografts were harvested, snap frozen in liquid nitrogen and stored at −70° C.

Radioimmunoassay (RIA) analysis for porcine C-peptide and rat insulin was conducted at sacrifice as follows. Insulin serum concentration and C-peptide concentration were determined with ¹²⁵I-labeled RIA. The RIA kits (Lineo Research, Inc., St. Charles, Mo., U.S.A.) utilized antibodies made specifically against the porcine C-peptide and rat insulin peptides. All samples were counted and the concentration of peptides was automatically calculated with a gamma counter (1282 Cumpagamma, LKB Instruments, Inc., Gaithersburg, Md., U.S.A.).

The following primary Abs were used for immunohistochemical analysis: W3/24 (anti-CD4; available from Pharmingen) W3/13 (anti-CD3; available from Pharmingen); OX-8 (anti-CD8; available from Pharmingen); NK1.2.3 (anti-NK cell; available from Pharmingen); OX-33 (anti-B cell; available from Serotec); ED1 (anti-CD68/macrophages; available from Serotec); and insulin from Dako. The immunohistochemical analysis was visualized by an avidin-biotin-peroxidase complex method and AEC as chromogen. In the case of a positive immunoreaction, a red-brown precipitate developed.

Results

Results of this study are presented in Table 1.

TABLE 1

| | Treat- | | | Toxic- | | ED1 | CD3 | | | OX33 | |
|---|---|---|---|---|---|---|---|---|---|---|---|
| grp | ment | n | PCP* | ity | Insulin | (CD68) | (Tcells) | CD8 | CD4 | (B cells) | NKRP-1 |
| 1 | control | 6 | <0.1 | none | 0/+ | ++ | ++/+++ | ++/+++ | +++ | ++ | ++/+++ |
| 4 | aCD40L | 3 | <0.1 | none | + | ++/+++ | +++ | +++ | +++ | ++/+++ | ++/+++ |
| 5 | aCD4 | 3 | 0 | none | ++(0) | ++ | +++(0) | ++ | +++ | ++ | ++ |
| 18 | Ha4/8 | 3 | | none | | | | | | | |
| 3 | FK | 3 | <0.1 | | ++ | + | ++ | + | ++ | + | +/++ |
| 13 | aCD40L + | 3 | 0.56 | none | ++/+++ | + | ++ | + | + | 0 | + |
| | aCD4 | | | | | | | | | | |
| 19 | Ha4/8 + | 3 | | none | | | | | | | |
| | aCD4 | | | | | | | | | | |
| 11 | FK + | 2 | <0.1 | | +++ | + | + | + | + | 0 | + |
| | aCD40L | | | | | | | | | | |
| 12 | FK + | 3 | 0 | | +++ | ++ | +/++ | + | + | + | + |
| | aCD4 | | | | | | | | | | |
| 16 | FK + | 3 | <0.1 | | +++ | + | + | + | + | 0 | + |
| | aCD40L + | | | | | | | | | | |
| | aCD4 | | | | | | | | | | |

*PCP levels are measured in ng/ml serum. Values below 0.9 or above 10.0 ng.ml fall off the standard curve. Reported value is the average value.
Key: For insulin staining, 0 = absent; + = single cell only; ++ = cell groups; and +++ = large cell groups; for degree of leukocytic infiltration (IHC), 0 = absent; + = single or few; ++ = moderate; +++ = plenty.

Immunohistochemical Evaluation

Control animals, i.e., untreated animals, had very few remaining insulin staining cells. There was a heavy infiltrate of macrophages, T and B cells. Many NK cells were also present. The graft was clearly rejected in a pattern described in the literature.

Animals treated by monotherapy, i.e., recipients that were administered solely AH.F5 or RIB 5/2, had single and small cell groups staining for insulin, respectively. Both drugs had an infiltration of mononuclear cells comparable to controls. Ha4/8 monotherapy had no effect in preventing rejection of the xenograft in recipients. In the FK506 monotherapy treated animals there was a significant difference in the pattern of infiltration. There was a fewer number of macrophages, CD3, CD4, CD8, B cells and NK cells than in the untreated controls.

Results from animals treated with double-therapy, i.e., with a combination of two antibodies are as follows. Animals administered a combination of anti-CD40L and anti-CD4 antibodies demonstrated prevention of the infiltration of ED1, CD3, CD4, CD8 and NK cells in the graft. In addition, B cells were completely absent from the graft. However, there was a diffuse perigraft infiltration consisting of a few macrophages (mainly on the capsular side of the graft) and T cells (mainly on the kidney/graft border). The graft itself was morphologically intact with strong staining for insulin in large cell groups. Animals treated with a combination of the control antibody Ha4/8 and anti-CD4 antibody demonstrated complete rejection. Treatment with a combination of FK506 and anti-CD40L antibodies also prevented rejection effectively. There were single or few infiltrating cells of all stained phenotypes, even fewer CD3 cells than in the anti-CD40L plus anti-CD4 double antibody treatment. The graft itself was morphologically intact with large cell groups with strong staining for insulin.

In animals treated with triple therapy, i.e., anti-CD40L, anti-CD4 together with FK506 antibodies, equal efficacy in preventing graft infiltration and rejection as achieved as with the combination of anti-CD40L and anti-CD4. The graft itself was morphologically intact with large cell groups, strongly staining for insulin.

Control animals increased on average 15% in weight. All antibody treated animals (alone or in combination, but without FK506) increased weight parallel to untreated controls. When FK506 was added, the animals failed to increase weight or lost a marginal amount, around 5%. This toxicity is significant, considering that the follow up was only 12 days and that the dose of FK506 (0.3 mg/kg BW) is equal or lower than in studies reporting no toxicity.

Control animals had very low levels of porcine C-peptide (PCP) in serum at 12 days. Animals treated with antibody monotherapy or FK alone were not different from controls. In the group treated with anti-CD40L and anti-CD4, there was 0.56 ng/ml PCP in serum, significantly higher than any other group.

### EXAMPLE 2

Combined Therapy with Non-Depleting Anti-CD4 and Anti-CD40L Prevents Islet Xenograft Rejection

Diabetes was induced in inbred male Lewis rats, weighing 250-270 grams, by the intravenous injection of streptozotocin, 55 mg/kg BW, nine days prior to islet cell transplantation. The measurement of glucose levels in the recipients revealed the onset of hyperglycemia (>400 mg glucose/dL) for three days prior to islet cell transplantation.

Islet cells from donor outbred female pigs, >2 years old, were prepared for transplant as described previously.

Recipient animals were anesthetized (Telazol, 0.20 mg/kg B.W., i.m.). Buprinorphine was administered to the recipient animals s.c. Transplantation was conducted as described above, during which 15,000 donor islet equivalents (IE) were injected together with anti-CD4 (RIB 5/2; 20 mg/kg BW, administered i.p. on day—1, 0, 1, 2, 3, 5, and then twice weekly) plus anti-CD40L (AH.F5; 12 mg/kg BW, administered i.p. on day—1, 0, 1, 7, and then twice weekly) combination therapy.

Following transplant, glucose levels were monitored daily for the first 15 days, and then every other day. PCP serum levels were monitored at the time of sacrifice and insulin extraction was performed as described above at the time of sacrifice. PCP monitoring was conducted by RIA as described above.

Insulin extraction was performed in all animals in this study at the time of sacrifice. Harvested pancreas tissue was snap-frozen in liquid nitrogen. To extract insulin, frozen tissue was homogenized in Ziegler Reagent, followed by sonication. After overnight incubation, the sample was buffered with 0.855 M Tris buffer. The sample was centrifuged for at 4° C. for 10 minutes at 2000×g. Then, the sample was aliquoted and stored at −70° C. pending RIA analysis, in which insulin serum concentration was determined with ¹²⁵I-labeled RIA. The RIA kits (Lineo Research, Inc., St. Charles, Mo., U.S.A.) utilized antibodies made specifically against rat insulin peptide. All samples were counted and the concentration of peptides was automatically calculated with a gamma counter (1282 Cumpagamma, LKB Instruments, Inc., Gaithersburg, Md., U.S.A.).

Immunohistochemical analysis was conducted as described previously.

Results

Results of this study are presented in Table 2.

TABLE 2

| | Location | Day | | | | | CD3 | | | OX33 | |
|---|---|---|---|---|---|---|---|---|---|---|---|
| Study | of | post | | Toxic- | | ED1 | (T | | | (B | |
| ID | Infiltrate | Tx | PCP* | ity | Insulin | (CD68) | cells) | CD8 | CD4 | cells) | NKRP-1 |
| XC- | perigraft, | 13 | 0.42 | None | +++ | + | 0 | + | + | 0 | ((+)) |
| 30 | scattered | | | | | | | | | | |
| | cells | | | | | | | | | | |
| XC- | perigraft, | 13 | pend. | None | +++ | (+) | (+) | ((+)) | ((+)) | ((+)) | ((+)) |
| 31 | scattered | | | | | | | | | | |
| | cells | | | | | | | | | | |
| XC- | | 18 | 0.24 | None | ++ | ++ | ++ | +++ | | + | + |
| 20 | | | | | | | | | | | |
| XC- | perigraft, | 18 | n.d. | None | ++ | n.d. | ++ | ++ | + | ++ | n.d. |
| 23 | 3 small | | | | | | | | | | |
| | dense foci | | | | | | | | | | |
| XC-24 | perigraft, | 18 | 0.37 | None | +++ | + | (+) | ((+)) | ((+)) | 0 | ((+)) |
| | scattered | | | | | | | | | | |
| | cells | | | | | | | | | | |
| XC-18 | perigraft, | 24 | 0 | None | + | ++ | +++ | ++ | + | + | + |
| | 3 small | | | | | | | | | | |
| | dense | | | | | | | | | | |
| | foci | | | | | | | | | | |
| XC-22 | perigraft, | 24 | 0.07 | None | +++ | ++ | +++ | +++ | +++ | + | + |
| | small | | | | | | | | | | |
| | dense | | | | | | | | | | |
| | foci | | | | | | | | | | |

*PCP levels are measured in ng/ml serum. Values below 0.9 or above 10.0 ng.ml fall off the standard curve. Reported value is the average value.
Key: For insulin staining, 0 = absent; + = single cell only; ++ = cell groups; and +++ = large cell groups; for degree of leukocytic infiltration (IHC), 0 = absent; + = single or few; ++ = moderate; +++ = plenty.

Graft morphology and the dynamics of the infiltrating cells in diabetic recipients receiving the combination therapy was studied. Animals were evaluated at day 13, 18 and 24 days post transplant. All animals were hyperglycemic at the time of sacrifice.

At day 13, there were very few cells infiltrating the graft (ED1, CD3, CD4, CD8, B cells, NK cells), identical to the results as described above. None of the cells infiltrated the graft, but instead remained in the perigraft area. The inflammatory cells were not clustered but rather were scattered around the graft.

At day 18, there was an increase of the number of ED1, CD3, CD8, B-cells, and NK cells in two (XC-20 and XC-23) of the three animals. However, the inflammatory cells did not enter the graft, but rather clustered around it. In the third animal, XC-24, the morphology of the graft appears as it did on day 13. The insulin stain revealed a large number of islet cell groups.

At day 24, the shift of cells to gather in clusters continued, with increased numbers of ED1, CD3, CD4, and NK positive cells. The graft itself was still not infiltrated. The inflammatory cells were found in small, dense foci. The small number of islet cells in XC-18 may be due to unbalanced sectioning of the graft or a suboptimal number of islet cells transplanted.

At days 13 and 18, the serum levels of PCP were comparable to those described in the previous example. However, at day 24, the serum levels were significantly lower, and not detectable in one animal (XC-18). The levels of PCP reflected graft function until day 18.

At no time in this experiment were the grafts infiltrated. Animals were sacrificed because graft rejection was suspected, rather than graft primary non-function (PNF). All animals in this experiment remained hyperglycemic until the time of sacrifice, with no difference in metabolic control in respect to either histology or level of serum PCP. It can be speculated that the mechanism with which the graft is indefinitely accepted with graft function is an active process, in which cytokines, e.g., IL-1, are secreted and inhibit the islets from maintaining glycemic control without harming them per se.

### EXAMPLE 3

Evaluation of Functional Graft Survival

Islet cells from donor outbred female pigs, >2 years old, were prepared for transplant as described above.

Streptozotocin-diabetic recipient rats (XC-11, XC-12, XC-35, XC-36 and XC-37) were anesthetized (Telazol, 0.20 mg/kg B.W., i.m.). Buprinorphine was administered to the recipient animals s.c. Transplantation was conducted as described above, during which the recipient animals received 7,500-15,000 donor islet equivalents (IE) and anti-CD4 (RIB 5/2) plus anti-CD40L (AH.F5) combination therapy as described above.

In this study, to protect the grafts from hyperglycemia, animals were given insulin injections (human regular and NPH insulin) during the first ten days after xenograft transplantation. Insulin was administered once daily on a sliding scale (Table 3).

TABLE 3

Insulin (U)

| Serum BGL (mg/dL) | Regular | NPH |
|---|---|---|
| less than 200 | 0 | 0 |
| 201-350 | 0 | 2 |
| 351-450 | 1 | 3 |
| more than 451 | 2 | 4 |

Following transplant, fed plasma glucose levels were monitored daily for the first 15 days or until the establishment of normoglycemia, after that, twice weekly. IVGTTs (0.5-1.0 g glucose/kg BW) were performed twice in each animal. The first test was performed around day 40-50 and the second was done either before nephrectomy or cessation of the immunosuppressive drug administration at day 100 (in two of the animals). Porcine C-peptide serum levels in response to glucose stimulation were assayed in each animal during the curative phase at around day 80 and rat C-peptide was assayed in response to glucose stimulation in some animals after nephrectomy. Xenograft morphology and beta cell/insulin content of the native pancreas were analyzed at the completion of the study.

Results

Results of this study are presented in Table 4.

TABLE 4

| | Location | sample | | | ED1 | | | | | |
|---|---|---|---|---|---|---|---|---|---|---|
| Study | of | tissue at | Toxic- | | (CD | CD3 | | | OX33 | |
| ID | Infiltrate | day | ity | Insulin | 68) | (Tcells) | CD8 | CD4 | (Bcells) | NKRP-1 |
| XC- | | 106 | none | ++++ | (+) | 0 | ((+)) | ((+)) | 0 | ((+)) |
| 11 | | | | | | | | | | |
| XC- | | 106 | none | ++++ | (+) | 0 | ((+)) | ((+)) | 0 | ((+)) |
| 12 | | | | | | | | | | |
| XC- | perigraft, | 100 | none | +++ | ++ | ++ | ++ | ++ | + | ++ |
| 35 | one | | | | | | | | | |
| | cluster | | | | | | | | | |
| XC- | T cell | 149 | none | 0 | ++ | +++ | ++ | +++ | + | (+) |
| 36 | dom- | | | | | | | | | |
| | inated rej. | | | | | | | | | |
| XC- | T cell | 132 | none | ((+)) | ++ | +++ | +++ | +++ | ++ | + |
| 37 | dom- | | | | | | | | | |
| | inated rej | | | | | | | | | |

Key: For insulin staining, 0 = absent; + = single cell only; ++ = cell groups; and +++ = large cell groups; for degree of leukocytic infiltration (IHC), 0 = absent; + = single or few; ++ = moderate; +++plenty.

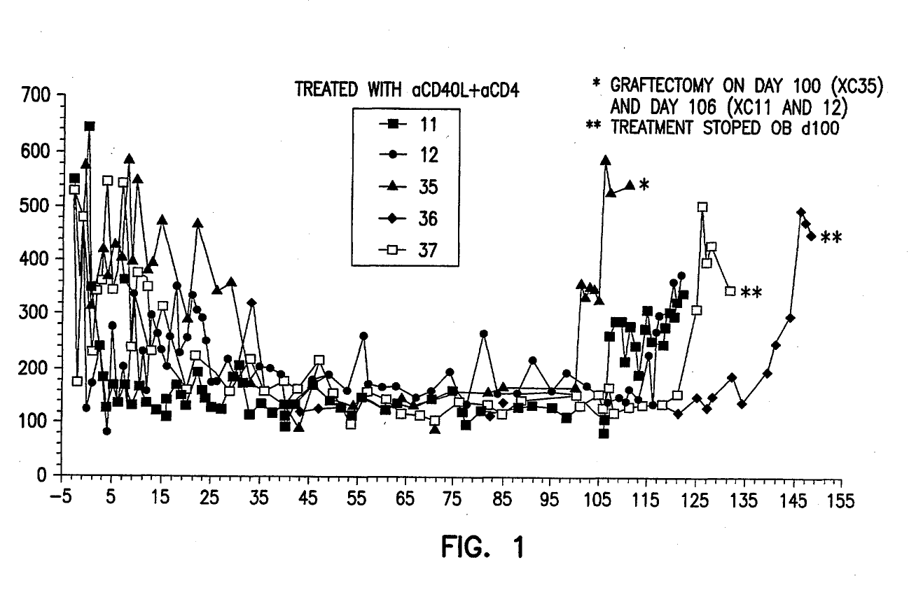

Normoglycemia was restored after 16±11.9 days and maintained for more than 100 days in diabetic rats receiving pig islet xenografts and combined therapy with RIB 5/2 and AH.F5 (FIG. 1). In fact, three animals demonstrated an initial period of PNF, and then turned normoglycemic.

All rats gained weight and showed no signs of toxicity.

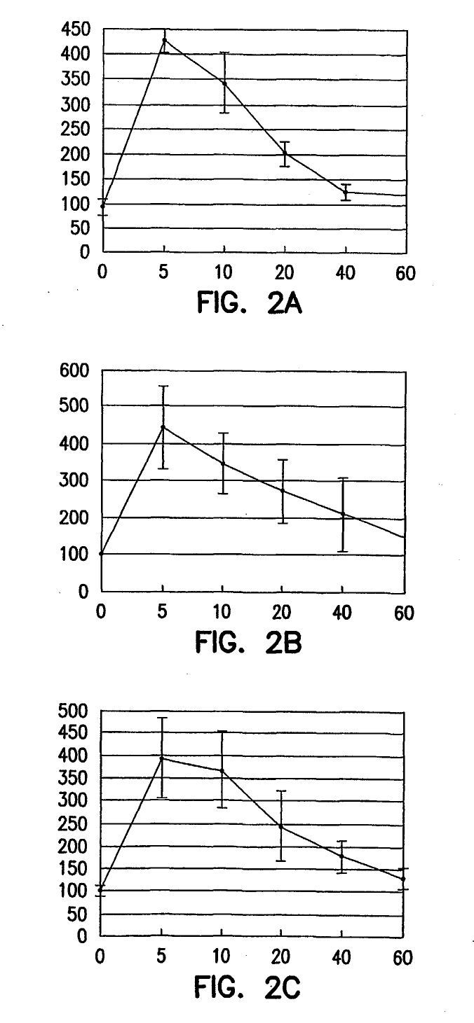

Glucose tolerance tests performed at day 40 and at day 100 showed adequate response to glucose challenge comparable to non-diabetic controls. IVGTTs performed in all animals at around day 50 and before graft nephrectomy or cessation of antibody administration at day 100. All showed normal lowering of plasma glucose in response to the stimulation and became normoglycemic at or before 40 minutes (FIG. 2).

Metabolic tests showed 46-185% increase in PCP and a return to normoglycemia within 40 minutes after an IV glucose challenge. Levels of PCP and porcine insulin (PI) were measured before and after glucose stimulation (0.5-1.0 g glucose/kg BW), measured at 0 and 20 minutes in serum. In all animals with xenograft, there was adequate response to glucose stimulation, showing that the xenografts were functioning (Table 5 and Table 6).

TABLE 5
Response in serum levels of porcine C-peptide to glucose stimulation.

| | | | Day | | Day | |
|---|---|---|---|---|---|---|
| | Day post | | post | | post | |
| | Tx | Δ % | Tx | Δ % | Tx | Δ % |
| XC-11 | 77 | 84 | 103 | 83 | 112 | 0 |
| XC-12 | 77 | 77 | 103 | 185 | 112 | 4 |
| XC-35 | 93 | 78 | | | | |
| XC-36 | 93 | 46 | | | | |
| XC-37 | 93 | 114 | | | | |

TABLE 6
Response in serum levels of porcine insulin to glucose stimulation.

| | | | Day | |
|---|---|---|---|---|
| | Day post | | post | |
| | Tx | Δ % | Tx | Δ % |
| XC-11 | 103 | 879 | 112 | −35 |
| XC-12 | 103 | 592 | 112 | −29 |
| XC-35 | 93 | 76 | | |
| XC-36 | 93 | 97 | | |
| XC-37 | 93 | 586 | | |

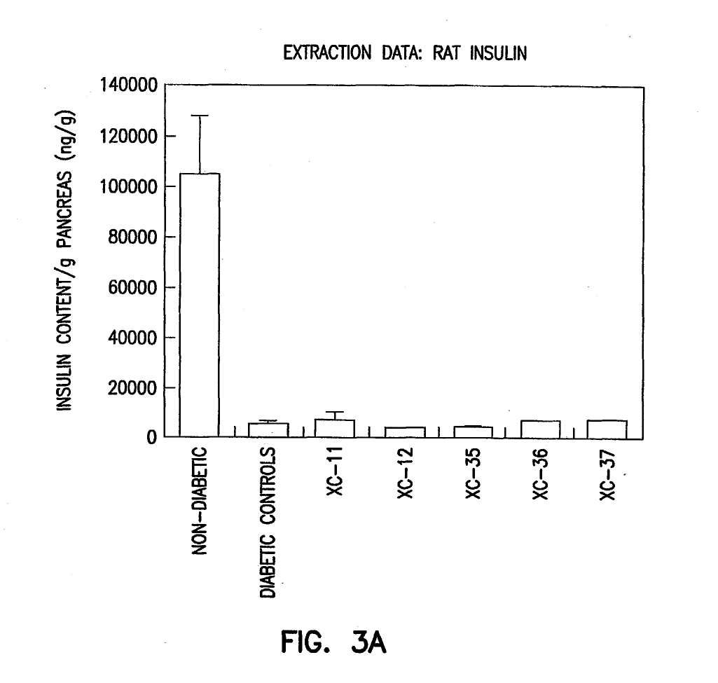

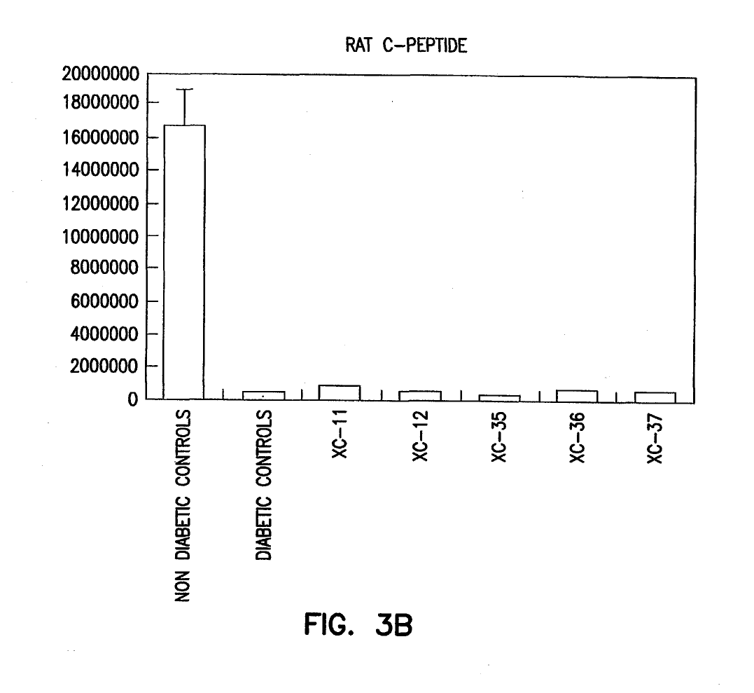

In all animals where drugs were ceased to be given at day 100 (XC-36 and XC-37), the animals remained normoglycemic (200 mg/dL) for 41 and 25 days respectively. Furthermore, XC-11 and XC-12 were nephrectomized on day 100 but stayed normoglycemic for 13 and 14 days respectively. To test whether the rat pancreas was regenerating functioning islets, insulin extraction tests on all rats were performed. Rat insulin extraction were performed on the sacrificed animals to ascertain that no function remained in the pancreas of the streptozotocin treated animals (FIGS. 3A and 3B). These results show a significant decrease of insulin and rat C-peptide (approximately 5%), consistent with histological findings.

Xenografts removed from three normoglycemic rats at day +100 revealed an abundance of insulin staining, rich neovascularization, and absence of infiltrating leukocytes. Rats returned to hyperglycemia 9.3±7.2 days after graft nephrectomy. The two animals in whom grafts were maintained after discontinuation of antibody therapy on day 100 remained normoglycemic for an additional 21 and 41 days. Histology showed a dense intra- and periislet infiltrate, dominated by CD4+ and CD8+ cells and a small number of CD68+ cells. The beta cell number and insulin content of the recipient's native pancreas were not different from diabetic, non-transplanted control animals.

Conclusion

In five out of five immunocompetent recipient Lewis rats, the combined modulation of signal 1, i.e., by the use of anti-CD40L antibody, with the blockade of signal 2, i.e., by the use of non-depleting anti-CD4 antibody, prevented islet xenograft rejection and reversed diabetes in the pig-to-rat model for more than 100 days in the absence of clinically evident toxicity. Metabolic and morphological studies proved xenograft function and survival.

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention.

What is claimed is:

1. A method for treating or preventing islet cell transplant rejection in a mammalian recipient, comprising administering to the recipient a combination of an antibody, fragment thereof, or mixture thereof that specifically binds to the CD40 ligand, and an antibody, fragment thereof, or mixture thereof that specifically binds to the CD4 receptor, in an amount of the combination effective to inhibit a T-cell mediated immune response in the recipient to the islet cell transplant.

2. The method of claim 1, wherein the recipient is a human.

3. The method of claim 1, wherein the islet cell transplant is xenogeneic to the recipient.

4. The method of claim 1, wherein the islet cell transplant is allogeneic to the recipient.

5. The method of claim 1, wherein the islet cell transplant comprises porcine cells.

6. The method of claim 1, wherein the combination is administered following transplantation.

7. The method of claim 1, wherein the combination is administered concurrently with transplantation.

8. The method of claim 1, wherein the antibody, fragment thereof, or mixture thereof that specifically binds to the CD40 ligand is monoclonal.

9. The method of claim 8, wherein the monoclonal antibody is 5c8.

10. The method of claim 1, wherein the antibody, fragment thereof, or mixture thereof that specifically binds to the CD40 ligand comprises MR1.

11. The method of claim 1, wherein the antibody, fragment thereof, or mixture thereof that specifically binds to the CD4 receptor is a non-depleting antibody.

12. The method of claim 11, wherein the non-depleting antibody is monoclonal.

13. The method of claim 1, further comprising administration of at least one anti-inflammatory or immunosuppressive drug.

14. The method of claim 13, wherein the anti-inflammatory or immunosuppressive drug is cyclosporin, cyclophosphamide, FK506, rapamycin, corticosteroids, mycophenolate mofetil, leflunomide, deoxyspergualin, azathioprine, or OKT-3.

15. The method of claims 1, wherein the amount is effective to induce immune tolerance in the recipient to the transplant.

16. A method for treating an autoimmune disease, comprising administering to a mammal afflicted with an autoimmune disease, a combination of an amount of at least one compound which specifically binds to the CD40 ligand, and an amount of at least one compound which specifically binds to the CD4 molecule, wherein the amounts are effective to inhibit a T-cell mediated immune response.

17. The method of claim 16 wherein the combination is comprised of a single chain antigen binding molecule, a small binding peptide or a mixture thereof.

18. The method of claim 16, wherein the combination is comprised of at least one antibody.

19. The method of claim 18, wherein the at least one antibody is a monoclonal.

20. The method of claim 16, wherein the autoimmune disease is Type I diabetes.
